(19) United States
(12) Patent Application Publication
(10) Pub. No.: US 2007/0148243 A1
(43) Pub. Date: Jun. 28, 2007

(54) Containment of a treatment agent in a body vessel

(76) Inventor: Brian L. Bates, Bloomington, IL (US)

Correspondence Address: DUNLAP, CODDING & ROGERS, P.C., P.O. BOX 16370, OKLAHOMA CITY, OK, 73113, US

(21) Appl. No.: 11/643,354

(22) Filed: Dec. 21, 2006

Related U.S. Application Data
(60) Provisional application No. 60/753,087, filed on Dec. 22, 2005.

(51) Int. Cl. A61K 9/14

(52) U.S. Cl. 424/484

(57) ABSTRACT

Medical devices useful for the containment of a treatment agent in a body vessel are described. The medical devices comprise a compressible plug having compressed and uncompressed diameters. Upon delivery to a treatment site in a body vessel, the compressible plug expands to engage the interior wall of the body vessel and provides a barrier that impedes fluid flow. The presence of the barrier facilitates containment of a treatment agent introduced into the body vessel at or near the point at which the medical device is deployed. Kits and methods are also described.

### FIELD OF THE INVENTION

The present invention relates to devices, kits, and methods for the embolization of blood vessels. Devices, kits, and methods according to the invention can be used in a variety of clinical situations, such as the treatment of varicose veins and venous disease generally.

### BACKGROUND OF THE INVENTION

The varying fluid environment within the vessels of the circulatory system can make it difficult to accurately deliver treatment agents to specific treatment sites in a vessel. Achieving a desired duration and extent of contact of the agent with the vessel while avoiding dilution can be difficult. Normal blood flow through a vessel works against containment of the agent in a discrete area.

Sclerosing agents are one type of treatment agent that may require site specific delivery. Sclerosing agents have been used in phlebology for many years and are enjoying somewhat of resurgence in popularity of late. These agents are frequently used to obliterate an entire section of a blood vessel when warranted, such as in the treatment of varicose veins. To be effective, the sclerosing agent must remain in contact with the vessel wall for a period of time and in an undiluted condition. Any loss of contact and/or change in concentration will decrease the effectiveness of the treatment.

One approach to mitigating the effects of blood flow is to isolate a portion of the vessel in which treatment is desired from the bloodstream, effectively removing that portion of the vessel from the flow pattern. One or more balloon catheters can be used to achieve such isolation, but these approaches are only partially effective because the catheters are not long-term implant devices and must be removed, which allows migration and dilution of the agent to occur. Any attempt to leave balloon catheters within a vessel for a prolonged period in an attempt to maintain the isolation or for other purposes can lead to other challenges including the potential for infection.

The prior art fails to teach satisfactory devices and methods for containing sclerosing agents in a portion of a body vessel. A need exists, therefore, for new and improved devices, kits, and methods useful in the containment of sclerosing and other treatment agents in a body vessel.

### SUMMARY OF EXEMPLARY EMBODIMENTS

Medical devices useful for containing a sclerosing agent within a particular body vessel or section of a body vessel are described.

A device according to one exemplary embodiment comprises a compressible plug formed of a bioremodellable material, such as an extracellular matrix (ECM) material. In exemplary embodiments, the plug is formed of an ECM material in the form of a foam or sponge, i.e., having open cells. In another exemplary embodiment, the plug is formed of a sheet of ECM material rolled into a plug formation, such as a cylindrical plug. In another exemplary embodiment, the plug is formed of a lyophilized material. In exemplary embodiments, the device includes barbs that facilitate anchoring of the device within a body vessel. In another exemplary embodiment, the plug is associated with an expandable support frame, such as a self-expandable stent.

Methods for sclerosing a body vessel are also described. The methods are useful in a variety of clinical situations, such as the treatment of varicose veins. One exemplary method comprises the steps of placing a compressible plug within the body vessel and introducing a sclerosing agent into the body vessel adjacent the compressible plug. In one exemplary method, a second compressible plug is placed within the body vessel to form a bordered section of the vessel within which a sclerosing agent can be contained.

Kits that can be used in a sclerosing treatment of a body vessel are also described. A kit according to one exemplary embodiment includes a sclerosing agent and a compressible plug formed of a bioremodellable material. Kits according to other exemplary embodiments include one or more delivery devices for placing the compressible plug(s) and/or sclerosing agent within the body vessel, such as a delivery sheath, dilator, and/or pusher.

Additional understanding of the invention can be obtained by reviewing the appended drawings and detailed description which illustrate and describe various exemplary embodiments of the invention.

### BRIEF DESCRIPTION OF THE DRAWINGS

FIG. 1 is a perspective view of a medical device according to an exemplary embodiment of the invention.

FIG. 2 is a schematic representation of a human leg and some of the body vessels associated with the leg.

FIG. 3 is a magnified view of area III referenced in FIG. 2.

FIG. 4 is a perspective view of a body vessel in which medical devices according to the invention have been placed.

FIG. 5 is an end view of a medical device according to another exemplary embodiment.

FIG. 6 is a perspective view of a sheet of material used to form the medical device illustrated in FIG. 5.

FIG. 7 is a perspective view of a sheet of material that can be used to form a medical device according to an exemplary embodiment of the invention.

FIG. 8 is a perspective view of a sheet of material that can be used to form a medical device according to an exemplary embodiment of the invention.

FIG. 9 is a perspective view of a medical device according to another exemplary embodiment of the invention.

FIG. 10 is a perspective view of a medical device according to another exemplary embodiment of the invention.

FIG. 11 is a schematic representation of a kit according to an exemplary embodiment of the invention.

FIG. 12 is a flow diagram of a method according to an exemplary embodiment of the invention.

### DETAILED DESCRIPTION OF EXEMPLARY EMBODIMENTS

The following detailed description and the appended drawings describe and illustrate exemplary embodiments of the invention for the purpose of enabling one of ordinary skill in the relevant art to make and use the invention. The description and drawings are not intended to limit the scope of the invention, or its protection, in any manner.

FIG. 1 illustrates a medical device 10 according to a first exemplary embodiment. The medical device 10 is a compressible plug formed of a bioremodellable material. As used herein, the term “plug” refers to a member capable of being inserted into a particular body vessel and substantially remaining in substantially the same location in the body vessel following insertion. The term does not require any particular dimensions and/or geometric configuration. Indeed, the particular dimensions and configuration chosen for any specific medical device according to the invention will depend on several considerations, such as the body vessel in which the device is intended to be implanted and the characteristics of the fluid and fluid flow normally associated with the body vessel. A cylindrical form is considered advantageous at least because, on expansion, an appropriately sized cylindrical plug can readily conform to body vessels having circular and ovoid cross-sectional profiles. Accordingly, this shape is illustrated in the various Figures. It is noted, however, that the cylindrical configuration is merely an exemplary configuration and is not required. For example, the medical device 10 may be conical or spherical in shape.

The medical device 10 can have any suitable length. The length chosen for a medical device according to a specific embodiment of the invention will depend on several considerations, including the nature of the vessel within which the device is intended for use, the nature of the material used in the device, and the nature of the sclerosing agent with which the device is intended for use. The overall length of the medical device 10 is believed to be one factor that contributes to the ability of the device to contain a particular sclerosing agent at a point within a body vessel. The inventor has determined that devices with a length of about 2 cm are suitable for use within a variety of body vessels and with a variety of sclerosing agents.

As a compressible member, the medical device 10 has a radially uncompressed diameter and a radially compressed diameter. For a device according to any specific embodiment of the invention, any suitable compressed and uncompressed diameters can be used. The specific diameters chosen will depend on several considerations, including the diameter and the elasticity of the body vessel within which the medical device 10 is intended for use. The medical device 10 should be capable of achieving a compressed diameter sufficient to allow the medical device 10 to be navigated through the vessel to a point of treatment, such as with an appropriate delivery device. Also, the medical device 10 should have an uncompressed diameter that allows the medical device 10 to engage the inner wall of the body vessel to substantially prevent its migration in the body vessel following deployment at a point of treatment. The inventor has determined that plugs having compressed diameters of about 3 mm and uncompressed diameters of about 16 mm are suitable for use in a variety of body vessels, including the great saphenous vein.

Any suitable material can be used in medical devices according to the invention. Bioremodellable materials are considered advantageous at least because of their well characterized biocompatibility and ability to substantially remodel to host tissue in a biological environment.

Bioremodellable material discussed herein can include extracellular matrix (ECM), for example pericardium, basement membrane, and amniotic membrane. One such ECM includes tissue submucosa, which further includes a small intestine submucosa (SIS), one type of tela submucosa. Tela submucosa is a multi-laminate structure, comprising the tunica submucosa, lamina muscularis mucosa, and the stratum compactum. The bioremodellable material herein can include the intestinal collagen layer described in U.S. Pat. No. 5,733,337 to Carr, et al. for TISSUE REPAIR FABRIC which is herein expressly incorporated by reference in its entirety for the purpose of describing exemplary bioremodellable materials. The tela submucosa can be made using the techniques described in U.S. Pat. No. 6,206,931 to Cook et al., for GRAFT PROSTHESIS, MATERIALS AND METHODS, which is herein expressly incorporated by reference in its entirety for the purpose of describing suitable techniques for preparing bioremodellable material. Other types of suitable bioremodellable materials include the materials described in the following disclosures, each of which is hereby expressly incorporated by reference in its entirety: U.S. Pat. No. 6,696,270 to Badylak, et. al. for GASTRIC SUBMUCOSAL TISSUE AS A NOVEL DIAGNOSIS TOOL; Liver tissue as described in U.S. Pat. No. 6,379,710 to Badylak, et al. for BIOMATERIAL DERIVED FROM VERTEBRATE LIVER TISSUE; U.S. Pat. No. 6,099,567 to Badylak, et al. for STOMACH SUBMUCOSA DERIVED TISSUE GRAFT; U.S. Pat. No. 5,554,389 to Badylak, et al. for URINARY BLADDER SUBMUCOSA DERIVED TISSUE GRAFT. Additionally, the medical device 10 may contain agents which promote retention of the compressed diameter. Such materials may include, for example, starch, cellulose, and sugars such as dextrose, or glycerin.

In exemplary embodiments, medical devices according to the invention are formed of a bioremodellable material configured as a foam or sponge, i.e., having open cells. The inventor has determined that a bioremodellable material in the form of a foam or sponge, i.e., having open cells, is a suitable material for use in veins due to the ability to absorb blood and swell to a swelled diameter. Such devices are particularly advantageous for use in the great saphenous vein. For example, the medical devices may be formed of sponge matrices comprising porous, three-dimensionally stable bodies formed from suitable biocompatible matrix materials as described in U.S. patent application Ser. No. 10/184,559 to Obermiller, et al. for POROUS SPONGE MATRICE MEDICAL DEVICES AND METHODS, which is herein expressly incorporated by reference in its entirety. This configuration is considered advantageous at least because the existence of open cells in the device facilitate the absorption of fluid, which can facilitate anchoring of the device in the body vessel following implantation. Gel forms are also considered advantageous. These materials swell upon the absorption and achieve a swelled diameter, which can be greater than the uncompressed diameter. The use of materials capable of achieving a swelled diameter can facilitate anchoring of the device within the body vessel. Any material capable of absorbing the appropriate body fluid can be used, and the specific material chosen for any particular medical device according to the invention will depend on several considerations, including the type of fluid normally associated with the body vessel in which the device is intended to be implanted.

Lyophilized materials, such as a lyophilized bioremodellable material, are also considered advantageous. These materials can be compressed in the sense that they have a relatively small diameter when dry and expand to a relatively larger diameter on contact with body fluids, such as blood. Lyophilized ECM materials, such as lyophilized SIS, are considered particularly advantageous at least because of their ability to remodel, the relative ease of their preparation, and their ready availability.

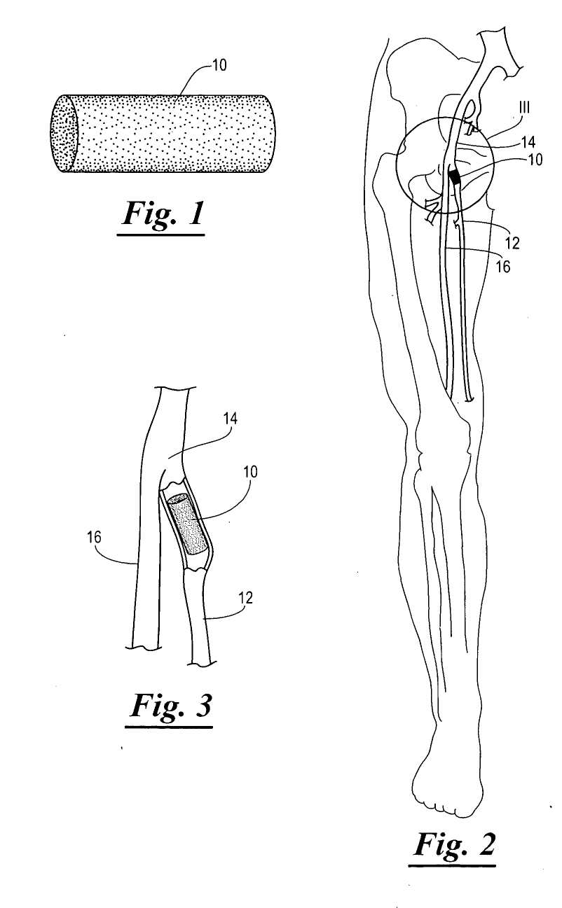

Devices according to the invention can be used in any body vessel within which it is desirable to contain an agent at or near a particular point of treatment. Devices according to the invention are particularly well suited for use with sclerosing agents, especially in the treatment of varicose veins. FIGS. 2 and 3 illustrate medical device 10 implanted within the great saphenous vein 12 in the leg of a human at a point distal to the junction 14 between the great saphenous 12 and femoral 16 veins. The medical device 10 has been deployed and allowed to expand substantially to the uncompressed diameter. The medical device 10 has engaged the inner wall of the great saphenous vein, which substantially prevents its migration within the vessel 12.

A treatment agent, such as a sclerosing agents or therapeutic agents, can be introduced into the vessel at or near the point at which the medical device 10 has been deployed. For example, proteins or other substances which promote clotting may be used. Alternatively, or in addition, any suitable sclerosing agent can be used. Examples of suitable sclerosing agents include morrhuate sodium, ethanolamine oleate, and tetradecyl sulfate.

The medical devices and methods described herein are advantageously used with treatment agents in foam form, including sclerosing foams. Treatment agents in foam form have a much larger surface area than in a fluid state. The smaller the cells or bubbles of foam are, the greater the surface area covered by the treatment agent. Foam form advantageously allows for an extended treatment surface area with only a small amount of treatment agent. Additionally, if a foam treatment agent, such as a sclerosing foam, is allowed to dissipate from a treatment site, contact between the treatment agent and the vessel wall is rapidly lost because of the relatively high ratio between the empty space of the foam, i.e., the cells or bubbles, and the agent.

Releasing the treatment agent directly into the medical device 10, such as with a needle or other means for injecting, is considered advantageous at least because it will likely cause additional swelling of the medical device 10, which facilitates anchoring of the medical device 10 in the vessel, and because it ensures containment of the treatment agent at the point at which the medical device 10 is implanted. The treatment agent can also be released at a point adjacent to the medical device 10. The treatment agent is expected to substantially be contained at the point of release with this approach, despite its release into the fluid within the vessel, because the medical device 10, once deployed, blocks fluid flow through the body vessel.

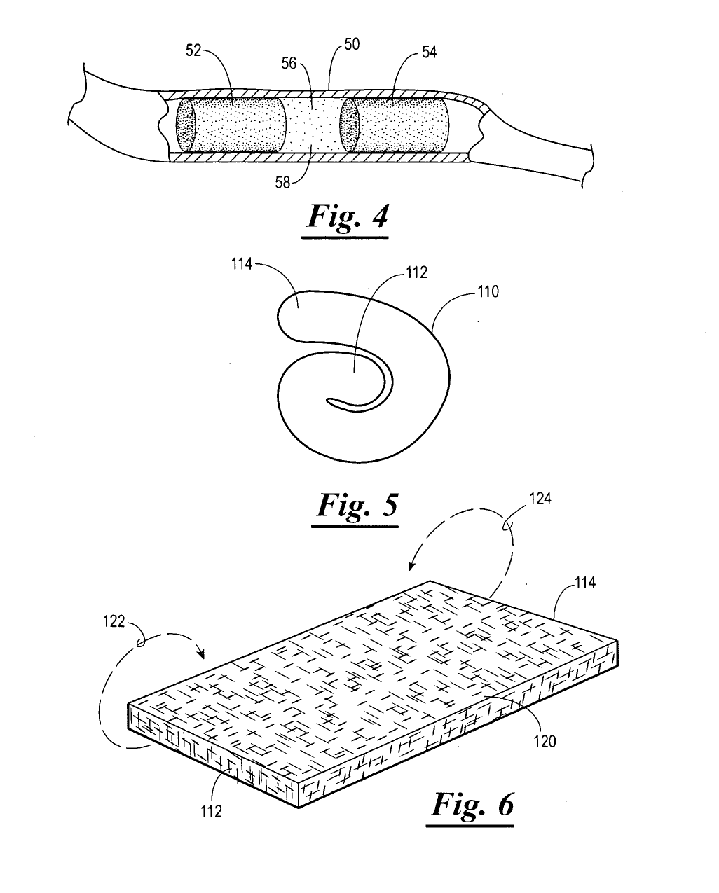

In some applications, it may be advantageous to isolate a portion of a body vessel using two medical devices according to the invention. FIG. 4 illustrates a body vessel 50 within which first 52 and second 54 medical devices have been deployed. As a result, a portion 56 of the body vessel 50 is completely isolated from fluid flow. A treatment agent 58, such as a sclerosing agent or therapeutic agent, can be released into this portion 56, either before, during, or after deployment of the second medical device 54. The treatment agent 58 can be released into the portion 50 by any suitable technique, including direct injection into the portion using a suitable delivery device, such as a syringe.

FIG. 5 illustrates a medical device 110 according to a second exemplary embodiment of the invention. In this embodiment, the medical device 110 is formed by folding a sheet 120 of bioremodellable material onto itself to create overlapping ends 1112, 114. As illustrated in FIG. 6, the device 110 can be formed by folding the first end inward, represented by arrow 122, and subsequently folding the second end inward, represented by arrow 124. A rolling manipulation of the sheet 120 could also be employed.

Following formation of the device 110, a constraining force can be applied so that the compressed diameter is achieved and/or maintained. The device 110 expands following deployment, i.e., when the constraining force is removed, by a partial unfolding of the overlapping ends 112, 114. Once exposed to fluid in the vessel, the bioremodellable material expands. The length and thickness of the sheet 120 is advantageously selected so that this swelling substantially blocks all fluid flow through the body vessel at the point of deployment. The length and thickness selected for a medical device according to a specific embodiment of the invention will depend on several considerations, including the degree to which the bioremodellable material is able to absorb the fluid within the body vessel.

In another embodiment, conventional shape memory material, including nickel-titanium alloys, ferromagnetic shape memory alloys, and/or the like, contained on or within the medical device 110 advantageously adopt a first curved configuration constraining the medical device in the compressed form. Activation of the shape memory material on or within the medical device causes the medical device 110 to adopt a second uncompressed configuration. Activation of the shape-memory material on or within the medical device 110 may occur through a suitable temperature change (e.g. from room temperature to body temperature), modification of the magnetic field, pressure modification, or the like depending on the specific type of shape-memory material in use.

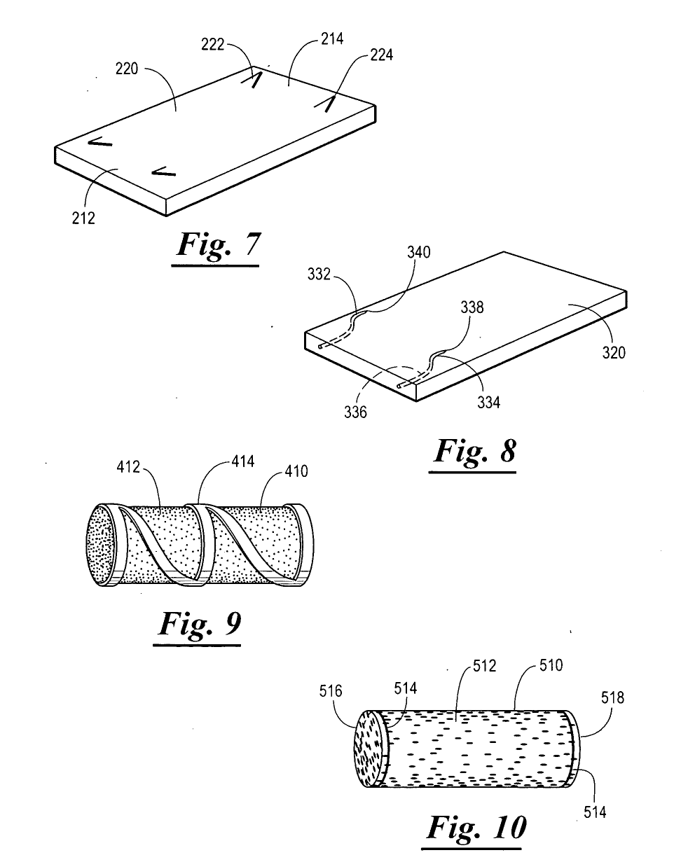

It may be desirable to provide additional structural features that facilitate anchoring of a medical device according to the invention at a point of treatment in a body vessel. Any suitable structural features can be used. FIGS. 7 and 8 illustrate exemplary structural features.

In FIG. 7, a sheet 220 of bioremodellable material includes integrally formed barbs 222. The barbs are advantageously relatively rigid as compared to the remainder of the sheet 220 and also advantageously define a point 224 adapted to pierce into and/or through the wall of a body vessel. The barbs may be formed from bioremodellable material similar to the material forming the sheet. The barbs may alternatively be formed of a separate material from the sheet such as steel wire. Any suitable process, technique, and/or treatment can be used to form the barbs 222, including chemical fixation of the portions forming the barbs 222.

Any suitable number, size, configuration and pattern can be used for the barbs 222. For example, in FIG. 7, a first set of barbs 222 at a first end 212 extend in a first direction and a second set of barbs 222 at a second end 214 extend in a second direction. When the first end 212 and the second end 214 overlap forming the medical device 200, the first series of barbs 222 at the first end 212 and the second series of barbs 214 at the second end anchor the medical device 200 within the body vessel.

In FIG. 8, a sheet 320 of bioremodellable material includes barbs 332, 334 composed of wire members. Each barb 332, 334 includes at least one portion 336 disposed within the thickness of the sheet 320 and at least one portion 338 that lies substantially adjacent the sheet 320. The portion 338 is advantageously spaced from the sheet 320 and defines a point 340 adapted to pierce into and/or through the wall of a body vessel. Any suitable wire material can be used to form the barbs 332, 334. Stainless steel is considered advantageous at least because of its well-characterized biocompatibility and ready availability. Any suitable number, size, configuration and pattern can be used for the barbs.

A support frame can also be included to enhance anchoring of a medical device according to the invention. FIG. 9 illustrates a medical device 410 according to an exemplary embodiment of the invention. The medical device 410 includes a compressible plug 412 and a support frame 414. The compressible plug 412 can have any suitable form as described above and can be attached to or simply associated with the support frame 414. Any suitable means for attaching the compressible plug 412 to the support frame 414 can be used if attachment is desired, including sutures, clips, adhesives, and other suitable means for attaching. Also, an interference fit can be employed. For example, the compressible plug 412 can be selected to have an uncompressed diameter that is larger than an uncompressed diameter of the support frame. This ensures that, upon expansion of both components, the compressible plug 412 will remain associated with the support frame 414.

A wide variety of support frames are known in the art, and any suitable support frame can be utilized. The support frame can provide a stenting function, i.e., exert a radially outward force on the interior vessel wall, but this function is not necessary and is considered optional.

The stent art provides numerous support frames acceptable for use in the invention, and any suitable stent can be used as the support frame. The specific support frame chosen will depend on numerous factors, including the vessel in which the device is being implanted, the axial length of the treatment site, the inner diameter of the vessel, the desired delivery method for placing the device, and others. Those skilled in the art can determine an appropriate support frame based on these various factors.

The support frame can be made from a variety of materials, and need only be biocompatible, or able to be made biocompatible, and provide a stenting function, if desired. Examples of suitable materials include, without limitation, stainless steel, nickel titanium (NiTi) alloys, e.g., nitinol, other shape memory and/or superelastic materials, polymers, and composite materials. Stainless steel and nitinol are particularly well-suited for use in the invention due to their biocompatibility, shapeability, and well-characterized nature.

The support frame can also have a variety of configurations, including braided strands, helically wound strands, ring members, consecutively attached ring members, tube members, and frames cut from solid tubes. The inclusion of open cells in the support frame is considered advantageous but not necessary.

Examples of suitable support frames include those described in U.S. Pat. No. 6,464,720 to Boatman et al. for a RADIALLY EXPANDABLE STENT; 6,231,598 to Berry et al. for a RADIALLY EXPANDABLE STENT; 6,299,635 to Frantzen for a RADIALLY EXPANDABLE NON-AXIALLY CONTRACTING SURGICAL STENT; 4,580,568 to Gianturco for a PERCUTANEOUS ENDOVASCULAR STENT AND METHOD FOR INSERTION THEREOF; each of which is hereby incorporated by reference in its entirety for the purpose of describing suitable support frames for use in medical devices according to exemplary embodiments of the invention.

The support frame 414 is advantageously attached to the compressible plug 412 and is advantageously disposed about the compressible plug 412, although other configurations are possible. For example, a support frame can be embedded in the compressible plug 412 and discrete attachment is considered optional.

Medical devices according to the invention can also include agents that enhance the visibility of the device using various imaging techniques. These features aid the user in properly placing the devices during a treatment protocol. FIG. 10 illustrates a medical device 510 that includes a compressible plug 512 and one or more markers 514. Each marker 514 is an agent or structural member that enhances the visibility of the device 510 in an imaging technique, such as radiography and venography. Any suitable marker can be used. Examples of suitable markers include opacifying agents such as bismuth and structural markers such as gold markers. The marker(s) 514 can be associated with the device 510 in any suitable manner and the manner chosen for a medical device according to a specific embodiment will depend on various considerations, including the nature of the marker 514 and the nature of the compressible plug 512. Opacifying agents are advantageously incorporated into foamed ECM materials, such as foamed SIS. Also, structural members, such as gold markers, are advantageously attached to an outer surface of a sheet of ECM material. These markers can also be advantageously associated with a support frame included in a particular medical device according to the invention.

No matter the form, the marker(s) 514 can be arranged in any suitable pattern in a medical device according to the invention, and the pattern chosen for a medical device according to a specific embodiment will depend on several considerations, including the overall length of the medical device and the nature of the point of treatment for which the medical device is intended. As illustrated in FIG. 10, markers 514 are advantageously associated with the medical device 510 at first 516 and second 518 ends. This placement allows the user to gain an understanding of the position of the entire medical device 510 during placement.

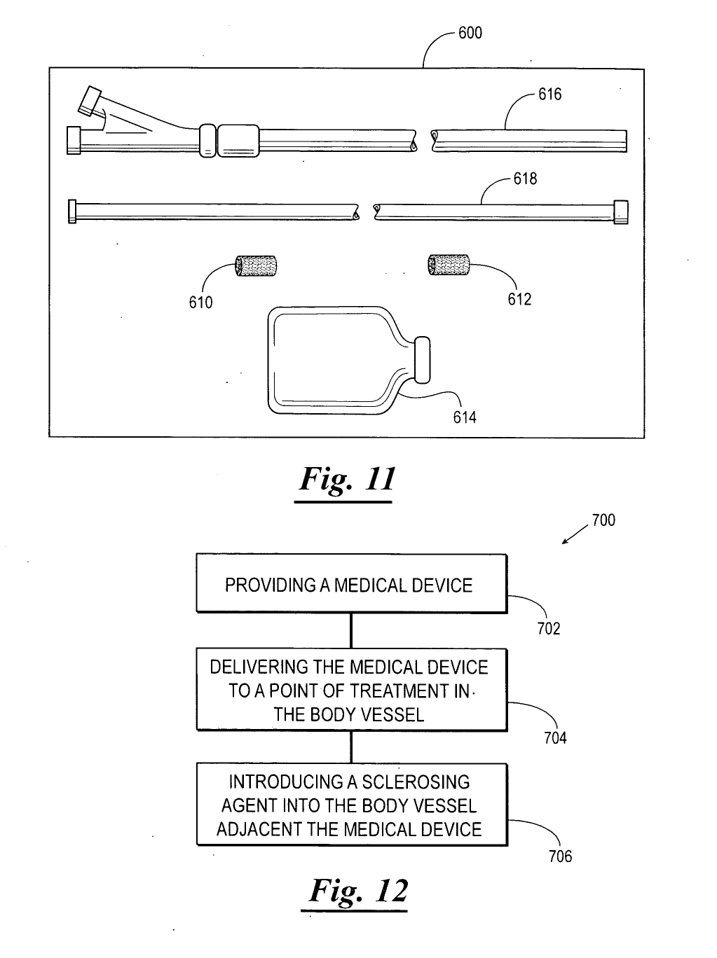

FIG. 11 illustrates a kit 600 according to an exemplary embodiment of the invention. The kit 600 includes first 610 and second 612 compressible plugs. A treatment agent 614, such as a sclerosing agent, is also included. A delivery device 616 is also included. An accessory device, such as a pusher 618, can also be included to facilitate implantation of the compressible plugs 610, 612.

While the kit 600 illustrated in FIG. 11 includes first 610 and second 612 compressible plugs, it is expressly understood that any suitable number of compressible plugs, including a single plug and three or more plugs, can be used in a kit according to the invention. The number of compressible plugs included in a kit according to a specific embodiment of the invention will depend on various considerations, including the nature of the body vessel and treatment site for which the kit is intended, the extent of the treatment for which the kit is intended, and other considerations that will be evident to skilled artisans.

The inclusion of the compressible plugs 610, 612 in a kit along with a delivery device 616 and a treatment agent is considered advantageous at least because it provides substantially all materials necessary to perform a particular treatment, such as a sclerosing treatment of a body vessel. The kits according to the invention also provide a convenient form for storing supplies in a health care provider facility and enable efficient reordering of treatment-related materials.

FIG. 12 illustrates a method 700 for sclerosing a body vessel according to the invention. A first step 702 comprises providing a medical device according to the invention in a body vessel. The medical device includes a compressible plug. Another step 704 comprises delivering the medical device to a point of treatment in the body vessel. Standard medical device delivery techniques can be used for this step, including minimally invasive techniques. For example, a delivery sheath can be placed in a body vessel such that the distal end of the sheath is disposed at or near a desired treatment site within the body vessel. Once the sheath is positioned in this manner, the dilator or inner member of the sheath can be removed through the proximal end and a medical device according to the invention can be loaded into the proximal end. A blunt tip pusher or other suitable device can then be used to advance the medical device through the sheath and, ultimately, out of the distal end such that it expands and becomes implanted at the treatment site. In another example, the medical device can be pre-loaded in the sheath and advanced in the body vessel as the sheath is advanced. Once positioned appropriately, the medical device can be forced out of the distal end of the sheath to affect deployment and implantation. Once deployed, the medical device contacts body fluid and is allowed to expand and engage the interior wall of the body vessel.

Another step 706 comprises introducing a sclerosing agent into the body vessel adjacent the medical device. The sclerosing agent can be introduced by any suitable delivery technique, including direct injection with a syringe.

In another exemplary method, a second medical device according to the invention is provided and delivered to a point in the body vessel that is spaced from the first medical device according to the invention, forming a bordered portion of the body vessel in which the sclerosing agent can be introduced and contained. The second medical device can be spaced from the first medical device by any suitable distance, and the distance chosen in any specific method according to the invention will depend on several considerations, including the nature of the body vessel, the nature of the sclerosing agent, and the desired treatment effect.

The sclerosing agent can be introduced into the body vessel prior to, during, and/or following delivery of the medical device(s) into the body vessel. It is considered advantageous to introduce the sclerosing agent into the body vessel after the medical device has been delivered. For example, in a method in which two medical devices are placed in a body vessel, the sclerosing agent can be introduced between two previously placed medical devices. Alternatively, a first medical device can be placed, then the sclerosing agent can be introduced, and finally a second medical device can be placed following introduction of the sclerosing agent.

The foregoing disclosure includes the best mode of the inventor for practicing the invention. It is apparent, however, that those skilled in the relevant art will recognize variations of the invention that are not described herein. Furthermore, while the invention is defined by the appended claims, the invention is not limited to the literal meaning of the claims, but also includes these variations.

I claim:

1. A medical device for containing a treatment agent at a treatment site within a body vessel, said medical device comprising a compressible plug formed of a bioremodellable material and having compressed and uncompressed diameters, the compressible plug adapted to engage an interior wall of said body vessel upon deployment at said treatment site.

2. The medical device according to claim 1, wherein the bioremodellable material comprises an extracellular matrix material.

3. The medical device according to claim 2, wherein the extracellular matrix material comprises small intestine submucosa.

4. The medical device according to claim 1, wherein the bioremodellable material includes open cells.

5. The medical device according to claim 1, further comprising at least one radiopaque marker associated with the compressible plug.

6. The medical device according to claim 1, wherein the compressible plug comprises at least one barb adapted to engage the interior wall of said body vessel.

7. The medical device according to claim 6, wherein the at least one barb is integrally formed by the compressible plug.

8. The medical device according to claim 6, wherein the at least one barb comprises a separate member associated with the compressible plug.

9. The medical device according to claim 6, wherein the at least one barb is rigid.

10. The medical device according to claim 6, wherein the barb is formed of bioremodellable material.

11. The medical device according to claim 6, wherein the barb is formed of stainless steel.

12. The medical device according to claim 6, wherein one portion of the at least one barb is disposed within the thickness of the compressible plug.

13. The medical device according to claim 1, wherein the compressible plug comprises a sheet having first and second ends folded into an overlapping configuration.

14. The medical device according to claim 13, wherein the sheet comprises at least one barb adapted to engage the interior wall of said body vessel.

15. The medical device according to claim 14, wherein a first portion of the at least one barb is substantially adjacent the sheet.

16. The medical device according to claim 15, wherein a second portion of the at least one barb defines a point adapted to engage the interior wall of said body vessel.

17. The medical device according to claim 1, further comprising a support frame disposed adjacent the compressible plug.

18. The medical device according to claim 17, wherein the support frame is disposed about the compressible plug.

19. The medical device according to claim 17, wherein the support frame comprises a stent.

20. The medical device according to claim 1, further comprising at least one radiopaque marker associated with at least one of the compressible plug and the support frame.

21. A kit for containing a treatment agent at a treatment site within a body vessel, comprising:
a plug formed of a bioremodellable material and having compressed and uncompressed diameters, the compressible plug adapted to engage an interior wall of said body vessel upon deployment at said treatment site;
a treatment agent adapted for injection adjacent the plug at said treatment site;
a delivery device adapted to deliver the plug to the treatment site.

22. A method for sclerosing a vein, comprising:
providing a medical device comprising a plug formed of a bioremodellable material;
delivering the medical device to a point of treatment in said vein;
exposing the medical device to a body fluid within said vein;
contacting the medical device to the body fluid;
allowing the medical device to expand and engage an interior wall of said body vessel;
introducing a sclerosing agent into said body vessel adjacent the medical device.
